(19) United States
(12) Patent Application Publication
(10) Pub. No.: US 2026/0224473 A1
(43) Pub. Date: Aug. 6, 2026

(54) INJECTABLE FORMULATION FOR PAIN REDUCTION INCLUDING BETA-NICOTINAMIDE MONONUCLEOTIDE AND METHOD OF PREPARING THE SAME

(71) Applicant: Si Ha KANG, Seoul (KR)

(72) Inventor: Si Ha KANG, Seoul (KR)

(21) Appl. No.: 19/335,487

(22) Filed: Sep. 22, 2025

(30) Foreign Application Priority Data
Jan. 31, 2025 (KR) 10-2025-0012341

(51) Int. Cl. A61K 9/00; A61K 9/08; A61K 9/19; A61K 31/706; A61K 47/02; A61K 47/18; A61K 47/26; A61K 47/42; A61P 29/02

(52) U.S. Cl. CPC A61K 9/0019; A61K 9/08; A61K 9/19; A61K 31/706; A61K 47/02; A61K 47/18; A61K 47/26; A61K 47/42; A61P 29/02; C12Y 302/01035

(57) ABSTRACT

The present invention relates to an injectable formulation for pain reduction, which includes β-NMN and a hyaluronidase and has a pH of more than 4 to 8, and a method of preparing the same.

### CROSS-REFERENCE TO RELATED APPLICATION

This application claims priority to and the benefit of Korean Patent Application No. 10-2025-0012341, filed on Jan. 31, 2025, the disclosure of which is incorporated herein by reference in its entirety.

### BACKGROUND

### 1. Field of the Invention

The present invention relates to an injectable formulation for pain reduction, which includes beta (β)-nicotinamide mononucleotide, and a method of preparing the same.

### 2. Discussion of Related Art

Globally, the increase in average life expectancy and improvements in living standards have intensified the demand for anti-aging or age-reversal. However, aging is the stage that follows maturity and is an inevitable process of all living organisms, occurring irreversibly over time and leading to death. Aging is influenced by genetic factors, lifestyle factors, and environmental factors. Although lifestyle and environmental improvements have occurred over the past centuries, the lack of a meaningful increase in maximum average life expectancy suggests that genetic factors play the most critical role in aging. Taking this into consideration, improvement in genetic factors is expected to delay an aging rate and achieve the natural maximum lifespan. Among the genetic factors of aging, the accumulation of DNA damage is recognized, and maintenance of DNA stability is an important approach to suppressing aging and extending lifespan.

Nicotinamide mononucleotide (NMN) was identified as a potential rejuvenation drug in 2016 after the research team of Prof. Shinichiro Imai (Washington, United States) confirmed a 16% lifespan extension in mice orally administered NMN. NMN, existing in the body, activates sirtuins, longevity-related genes, and is reduced with aging. NMN is a nucleotide that is naturally generated through a reaction between a nucleoside containing ribose and nicotinamide (NAM) and a phosphate group. It has two isomeric forms, α-NMN and β-NMN, with the β isomer being the active form.

Such β-NMN has been marketed in the United States as an oral health supplement and nutraceutical. Since the oral formulation must pass through various digestive organs, only a part of the ingredient is absorbed, resulting in the need for a higher dosage. While sublingual administration exhibits the maximum 30% bioavailability, there is a problem of considerable interindividual variability depending on the characteristics of oral administration and the residence time under the tongue. In addition, the dosage of β-NMN nutraceuticals currently used in the United States is set at up to 1250 mg per day; however, due to the high cost of β-NMN, there is a limitation in consuming large amounts daily.

Compared to such oral administration, the administration of an injectable formulation is an optimal method to deliver an exact dose rapidly and in a well-controlled manner, thereby achieving systemic effects. In addition, such injectable formulations have the advantage of having the same effect with a smaller dose than oral administration. However, in this case, the stability and safety of injectable formulations should be given important consideration.

Therefore, in development of injectable formulations containing β-NMN, there is a demand for improving stability and safety. Particularly, in subcutaneous or intramuscular administration of injectable formulations, optimization studies are needed to minimize pain induction.

### SUMMARY OF THE INVENTION

The present invention is directed to providing an injectable formulation for pain reduction, which includes β-NMN and a hyaluronidase, and has a pH of more than 4 to 8.

However, technical problems to be solved in the present invention are not limited to the above-described problems, and other problems which are not described herein will be fully understood by those of ordinary skill in the art from the following descriptions.

According to one aspect of the present invention, there is provided an injectable formulation for pain reduction in injection, which includes β-NMN and a hyaluronidase, and has a pH of more than 4 to 8.

The injectable formulation may have a pH of 5 to 7.

The osmotic pressure of the injectable formulation may be 800 mOsmol/kg to 1,600 mOsmol/kg.

The injectable formulation may be used for subcutaneous or intramuscular administration.

The injectable formulation may further include one or more stabilizers selected from the group consisting of monosaccharides, disaccharides, and sugar alcohols.

The stabilizers may be i) one or more selected from the group consisting of mannitol, sorbitol, lactose, xylitol, trehalose, sucrose, maltose, and glucose, or include ii) mannitol as a first stabilizer, and one selected from the group consisting of xylitol, trehalose, sucrose, maltose, and glucose as a second stabilizer.

The injectable formulation may further include one or more alkalizing agents selected from the group consisting of sodium hydroxide (NaOH), calcium hydroxide (Ca(OH)₂), potassium hydroxide (KOH), tromethamine, and sodium bicarbonate, or hydrochloric acid (HCl).

In the injectable formulation, the β-NMN may be administered daily at a dose of 0.5 mg/kg to 500 mg/kg.

At 5, 30, 60, 120, and 360 minutes after administering the injectable formulation, blood samples are collected and analyzed using a NAD+/NADH assay kit. As a result, it can be confirmed that i) the mean blood NAD+ concentration shows an increase to 200% to 400% at each time point, compared to placebo administration, and ii) the mean blood NADH concentration shows an increase to 150% to 300% at each time point, compared to placebo administration.

According to another aspect of the present invention, there is provided a method of preparing an injectable formulation, which includes: (a) preparing a solution including β-NMN and a hyaluronidase; (b) preparing a lyophilized formulation by lyophilizing the solution; and (c) applying an infusion solution to the lyophilized formulation, wherein the injectable formulation has a pH of more than 4 to 8.

In (b), the water content in the lyophilized formulation may be 5 (w/w) % or less.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows the result of visually observing the appearance of injectable formulations (SC formulations) prepared using β-NMN and a lyophilized hyaluronidase formulation according to Comparative Example 1 and Examples 1 to 3, after storing them at 25° C. for 8 weeks; and

FIG. 2(A) shows the result of assessing blood NAD+ concentrations in subcutaneous administration of the injectable formulation for pain reduction (SC formulation) containing β-NMN and a hyaluronidase, prepared in Example 1, and FIG. 2(B) shows the result of assessing blood NADH concentrations in subcutaneous administration of the injectable formulation for pain reduction (SC formulation) containing β-NMN and a hyaluronidase, prepared in Example 1.

### DETAILED DESCRIPTION OF EXEMPLARY EMBODIMENTS

The present inventors confirmed that an optimal lyophilized formulation (particularly, a lyophilized formulation maintaining optimal pH and osmotic pressure conditions) was used in development of injectables containing β-NMN and a hyaluronidase, and was able to improve stability and safety and especially minimize pain induction, thus completing the present invention.

Hereinafter, the present invention will be described in detail.

### Injectable Formulation for Pain Reduction

The present invention provides an injectable formulation for pain reduction, which includes β-NMN and a hyaluronidase, and has a pH of more than 4 to 8.

In the “injectable formulation for pain reduction” used herein, pain reduction means a decrease in an unpleasant sensation associated with substantial and potential tissue damage, and such pain may be caused by a syringe needle, and occur in a local area around the tissue penetrated by the needle.

First, the injectable formulation for pain reduction according to the present invention includes β-NMN.

Specifically, the β-NMN is also called β-NMN, and its specific structure is as follows:

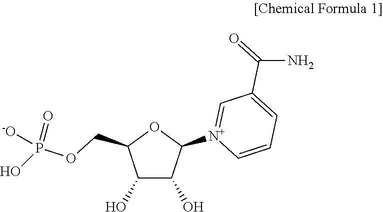

The β-NMN is a NAD+ precursor, which may be converted into NAD+ in the body and has a variety of effects, including anti-aging, increased energy, improved cardiovascular health, protection of cognitive function, enhanced metabolism, and reduced inflammation and oxidative stress.

Accordingly, the β-NMN in the injectable formulation for pain reduction according to the present invention may be maintained at a concentration of 1 mg/mL to 1,000 mg/mL, and preferably 100 mg/mL to 500 mg/mL, but the present invention is not limited thereto. For these reasons, the injectable formulation for pain reduction according to the present invention is suitable for, particularly, subcutaneous or intramuscular administration.

In addition, the injectable formulation for pain reduction according to the present invention may further include a hyaluronidase. Such a hyaluronidase is a substance added to allow the β-NMN to more effectively function in the body, and may have a synergistic effect with the β-NMN to improve the NAD+/NADH ratio in the body.

Specifically, hyaluronidases are a large family of enzymes that degrade hyaluronic acid. The hyaluronic acid is the key ingredient of the extracellular matrix, and the main component of the interstitial barrier. The hyaluronidase catalyzes the hydrolysis of the hyaluronic acid, thereby lowering the viscosity of the hyaluronic acid, resulting in increased tissue permeability and diffusion. Accordingly, the hyaluronidase is used as a spreading agent or dispersion agent, in conjunction with other reagents, drugs, or proteins, to speed up dispersion and delivery.

In other words, by promoting (or catalyzing) the hydrolysis of hyaluronic acid, the hyaluronidase facilitates the effective diffusion, delivery, and action of the β-NMN within the body.

In addition, the hyaluronidase may include a hyaluronidase derived from any non-human origin, or a human-derived hyaluronidase.

Here, non-human-derived hyaluronidases may include hyaluronidases derived from rodents, dogs, cats, rabbits, birds, cattle, sheep, pigs, horses, fish, frogs, fungi, leeches, other parasites, and crustaceans. For example, non-human-derived hyaluronidases may include hyaluronidases derived from cattle, hornets, honeybees, white-faced hornets, Asian giant hornets, mice, pigs, rats (laboratory mice), rabbits, sheep, orangutans, Philippine monkeys, guinea pigs, Staphylococcus aureus, Streptococcus, and Clostridium perfringens.

In addition, human-derived hyaluronidases may include HYAL1, HYAL2, HYAL3, HYAL4, PH20, or variants thereof.

The content of the hyaluronidase in the injectable formulation for pain reduction according to the present invention may be 5 IU/mL to 1,500 IU/mL. The hyaluronidase in the injectable formulation for pain reduction according to the present invention may be maintained at a concentration of 50 IU/mL to 1,000 IU/mL, but the present invention is not limited thereto.

For the injectable formulation for pain reduction according to the present invention, an optimal pH condition must be maintained. Accordingly, the optimal pH condition is preferably a pH of more than 4 to 8, more preferably a pH of 5 to 7, and most preferably pH 6 (±0.5), but the present invention is not limited thereto. Therefore, the injectable formulation for pain reduction has an advantage of considerably reducing pain in injection.

In addition, the osmotic pressure of the injectable formulation for pain reduction according to the present invention may be 800 mOsmol/kg to 1,600 mOsmol/kg, preferably 800 mOsmol/kg to 1,300 mOsmol/kg, and more preferably 1,000 mOsmol/kg to 1,200 mOsmol/kg, but the present invention is not limited thereto. Such osmotic pressure may be regulated by adding an osmotic regulator as needed, and the content of the osmotic regulator must be limited not to excessively reduce osmotic pressure.

Particularly, as the injectable formulation for pain reduction according to the present invention is prepared from a lyophilized formulation, there may be almost no change in β-NMN content during storage at 25° C. for 8 weeks, and thus the injectable formulation for pain reduction according to the present invention exhibits excellent stability and safety. In the preparation of the injectable formulation for pain reduction, when a liquid formulation is used without lyophilizing, a change in β-NMN content is greatly increased in storage at 25° C. for 8 weeks, and therefore there is a problem in that the stability and safety of the injectable formulation for pain reduction are greatly degraded.

Meanwhile, the injectable formulation for pain reduction according to the present invention may further include a stabilizer to prevent the hydrolysis of the β-NMN. As the stabilizer, those that are well known in the art may be employed, and include preferably one or more selected from the group consisting of monosaccharides (glucose, etc.); disaccharides (trehalose, sucrose, lactose, maltose, isomaltose, etc.); and sugar alcohols (mannitol, xylitol, sorbitol, maltitol, erythritol, lactitol, etc.), and more preferably sugar alcohols (mannitol, xylitol, sorbitol, maltitol, erythritol, lactitol, etc.), but the present invention is not limited thereto.

Specifically, the stabilizer may include i) one or more selected from the group consisting of mannitol, sorbitol, lactose, xylitol, trehalose, sucrose, maltose, and glucose, or ii) a combination of mannitol as a first stabilizer and one or more selected from the group consisting of xylitol, trehalose, sucrose, maltose and glucose as a second stabilizer.

For example, the stabilizer may include mannitol alone as the first stabilizer. Alternatively, the stabilizer may include mannitol as the first stabilizer, in combination with xylitol, trehalose, sucrose, maltose, or glucose as the second stabilizer. Alternatively, the stabilizer may be, instead of mannitol, sorbitol, lactose, xylitol, trehalose, sucrose, maltose, or glucose alone, or in combination with xylitol and sucrose as the first stabilizer.

More specifically, the content of the stabilizer may be 10 wt % to 500 wt %, 20 wt % to 50 wt %, or 30 wt % to 40 wt % with respect to the β-NMN content, but the present invention is not limited thereto.

In addition, the injectable formulation for pain reduction according to the present invention may further include a pH adjuster to maintain the optimal pH condition, and may further include one or more alkalizing agents selected from the group consisting of sodium hydroxide (NaOH), calcium hydroxide (Ca(OH)₂), potassium hydroxide (KOH), tromethamine, and sodium bicarbonate, or hydrochloric acid (HCl). Here, the alkalizing agent is preferably one or more selected from the group consisting of sodium hydroxide (NaOH), calcium hydroxide (Ca(OH)₂), potassium hydroxide (KOH), tromethamine, and sodium bicarbonate, and more preferably one or more selected from the group consisting of sodium hydroxide (NaOH), calcium hydroxide (Ca(OH)₂), and potassium hydroxide (KOH) to suppress the generation of insoluble fine particles and ensure stability, but the present invention is not limited thereto. For example, to satisfy the optimal pH condition of the injectable formulation for pain reduction according to the present invention, the alkalizing agent may be used in combination with hydrochloric acid (HCl), or hydrochloric acid (HCl) alone may be used without the alkalizing agent.

Optionally, the injectable formulation for pain reduction according to the present invention may further include an additive such as an osmotic regulator or a surfactant as long as it does not change physical properties. As the osmotic regulator, hydroxy-gamma-cyclodextrine (HP-gamma-cyclodextrine), or captisol may be used, and to avoid excessively lowering the osmotic pressure, the content of the osmotic regulator may be 10 wt % to 20 wt %, preferably 10 wt % to 15 wt %, with respect to the β-NMN content, but the present invention is not limited thereto. In addition, as the surfactant, a polysorbate-based surfactant may be used, and its content may be 1 wt % to 5 wt %, preferably 1 wt % to 2 wt %, with respect to the β-NMN content, but the present invention is not limited thereto.

In addition, an injectable lyophilized formulation for injection according to the present invention needs to be diluted in an infusion solution to regulate the concentration of the β-NMN. Specifically, the infusion solution may be water for injection, or further include an isotonic agent such as sodium chloride or glucose in water for injection (e.g., 0.1 to 2.0% physiological saline injection, or 1 to 10% glucose injection or lactose-added Ringer's solution). Accordingly, the β-NMN in the injectable formulation for pain reduction according to the present invention may be maintained at a concentration of 1 mg/mL to 1,000 mg/mL, preferably 100 mg/mL to 500 mg/mL, but the present invention is not limited thereto. Therefore, the injectable formulation for pain reduction according to the present invention is suitable for subcutaneous or intramuscular administration.

Meanwhile, the injectable formulation for pain reduction according to the present invention may be for subcutaneous or intramuscular administration, and preferably for subcutaneous administration, but the present invention is not limited thereto. Unlike an oral formulation, such an injectable formulation can be rapidly administered at an exact dose and well controlled, thereby achieving systemic effects.

However, for the direct injection of an injectable formulation to a blood vessel, stability and safety must be primarily considered. Particularly, unlike intravenous administration, subcutaneous or intramuscular administration of an injectable formulation enables fast administration in an emergency, and particularly, subcutaneous administration has the advantage that patients are able to administer a drug by themselves-administer. However, in these administration methods, patient reluctance caused by severe pain has become an issue, so considerations to reduce pain are important. The injectable formulation for pain reduction according to the present invention uses a lyophilized formulation maintaining optimal pH and osmotic pressure conditions, which is advantageous in minimizing pain induction.

In the injectable formulation for pain reduction according to the present invention, the dose of the β-NMN may be administered in an amount of 1/20 to ½, and preferably, 1/20 to ¼ of the amount determined to be therapeutically effective in oral administration. Despite administration at such a small dose, the β-NMN may exert an effect equal to or greater than that of the regular dose, and exhibit an immediate drug-release effect in the body.

That is, in the injectable formulation for pain reduction according to the present invention, the β-NMN may be administered daily at a dose of 0.5 mg/kg to 500 mg/kg.

Blood samples were collected at 5, 30, 60, 120, and 360 minutes after administering the injectable formulation for pain reduction according to the present invention (after administering the β-NMN at 500 mg/kg/day for 3 days) and analyzed using a NAD+/NADH assay kit, revealing that i) the mean blood NAD+ concentrations may show an increase to 200% to 400% (particularly, 250% to 350%) at each time point, and ii) the mean blood NADH concentrations show an increase to 150% to 300% (particularly, 150% to 250%) at each time point, compared to placebo administration.

Particularly, blood samples were collected at 5, 30, and 60 minutes after administering the injectable formulation for pain reduction according to the present invention (after administering the β-NMN at 500 mg/kg/day for 3 days), and analyzed using a NAD+/NADH assay kit, revealing i) a 350% to 450% increase in the mean blood NAD+ concentration at the earlier time points, and ii) a 250% to 350% increase in the mean blood NADH concentration at the earlier time points, compared to placebo administration.

In addition, a blood sample was collected at five minutes after administering the injectable formulation for pain reduction according to the present invention and analyzed using a NAD+/NADH assay kit, revealing i) a 500% to 550% increase in the blood NAD+ concentration compared to placebo administration, and ii) a 450% to 500% increase in the blood NADH concentration, compared to placebo administration.

That is, when the injectable formulation for pain reduction according to the present invention is administered, it can be seen that the increments in the mean and maximum blood NAD+ concentrations are comparatively larger than those of placebo administration. Particularly, the subcutaneous administration of the injectable formulation for pain reduction according to the present invention can optimize the NAD+/NADH ratio in the body, and show an excellent effect in anti-aging.

### Method of Preparing Injectable Formulation for Pain Reduction

The present invention provides a method of preparing an injectable formulation for pain reduction, which includes: (a) preparing a solution containing β-NMN and a hyaluronidase; (b) preparing a lyophilized formulation by lyophilizing the solution; and (c) adding an infusion solution to the lyophilized formulation, wherein the injectable formulation for pain reduction has a pH of more than 4 to 8.

First, the present invention includes operation (a) of preparing a solution containing β-NMN and a hyaluronidase.

First, the solution contains β-NMN. Since the β-NMN has been described above, repetitive description will be omitted. In addition, to exhibit a synergistic effect with the β-NMN improving a NAD+/NADH ratio in the body, the solution contains a hyaluronidase. Since the hyaluronidase has also been described above, repetitive description will be omitted.

Optionally, a stabilizer may be further contained to prevent the hydrolysis of the β-NMN. Since the specific types of stabilizers have been exemplified, repetitive description will be omitted.

As needed, an additive such as a pH adjuster (an alkalizing agent and/or HCl), an osmotic regulator, or a surfactant may be additionally contained. Since the specific types of additives have been exemplified, repetitive description will be omitted.

In this solution, nitrogen-substituted water for injection may be used as a solvent. The nitrogen-substituted water for injection may be prepared by a process known in the art, and has the benefit in cake formation following lyophilizing after oxygen is substituted with nitrogen through dissolution and filling processes.

Next, the present invention includes operation (b) of preparing a lyophilized formulation by lyophilizing the solution.

Before the lyophilizing operation, a stabilizing operation may precede, which may be performed at 1° C. to 10° C. for 10 minutes to 200 minutes under atmospheric pressure.

The lyophilizing operation is divided into a freezing operation and a drying operation, wherein the freezing operation may be performed at −100° C. to −1° C. (preferably −80° C. to −5° C.) for 100 minutes to 1000 minutes under atmospheric pressure. To minimize bubble generation, the freezing operation is performed in two steps: The first step may be performed at −10° C. to −1° C.; and the second step may be performed at −100° C. to −50° C. Meanwhile, the drying operation may be performed at 5° C. to 50° C. (preferably 10° C. to 25° C.) for 500 minutes to 3000 minutes under 0.1 Pa to 1 Pa. To form a cake with minimized water content, the drying operation may be performed in two steps: The first step may be performed at 5° C. to 15° C.; and the second step may be performed at 20° C. to 50° C.

The lyophilized formulation has minimized water content, which may be 5 (w/w) % or less, and preferably 3 (w/w) % or less, but the present invention is not limited thereto. The lyophilized formulation may be formed in a cake with minimized water content.

Next, the present invention includes operation (c) of adding an infusion solution to the lyophilized formulation. Accordingly, the injectable formulation for pain reduction according to the present invention may be finally prepared with a pH of more than 4 to 8.

The infusion solution is for adjusting the concentration of the β-NMN. Since the specific details have been provided above, repetitive description will be omitted. Accordingly, the β-NMN in the injectable formulation for pain reduction according to the present invention may be maintained at a concentration of 1 mg/mL to 1,000 mg/mL, and preferably 100 mg/mL to 500 mg/mL, but the present invention is not limited thereto. Therefore, this case is suitable for, particularly, the subcutaneous or intramuscular administration.

As seen from the above, the injectable formulation for pain reduction according to the present invention includes β-NMN and a hyaluronidase, and has a pH of more than 4 to 8, and therefore is advantageous for improving stability and safety and especially minimizing pain induction.

In addition, the injectable formulation for pain reduction according to the present invention may have an optimized NAD+/NADH ratio in the body, and may exhibit an excellent effect in anti-aging.

Hereinafter, preferred examples will be presented to allow the present invention to be better understood. However, the following examples are merely provided in order for the present invention to be more easily understood, and the content of the present invention is not limited by the following examples.

### EXAMPLES

### Preliminary Examples 1 to 8: Freeze-Dried Formulations Containing β-NMN and Hyaluronidase and Preparation of Reconstituted Solutions Thereof

900 mg of β-NMN and 300 IU of a hyaluronidase (Ningbo Zlinzyme Biosciences, China) were added to 2.7 mL of nitrogen-substituted water for injection, stirred and constituted for 60 minutes. Subsequently, a stabilizer, an osmotic regulator, and a surfactant were optionally added to constitute for 30 minutes, and then an alkalizing agent or HCl was added q.s. as a pH adjuster for pH adjustment. The solution was collected up to 3 mL. The collected solution was filtered through a 0.2 μm cellulose acetate (CA) filter. The filtered solution was put into a 20 mL vial, and lyophilized using a lyophilizing apparatus (JABA Lyoph-Pride LP20), thereby preparing a lyophilized formulation for β-NMN injection (water content≤5 (w/w) %) as shown in Table 1. Here, lyophilizing was performed by 1) a loading process at 5° C. under atmospheric pressure; 2) a stabilization process at 5° C. for 120 minutes under atmospheric pressure; 3) a freezing process at −25° C. for 720 minutes under atmospheric pressure; 4) a primary drying process at 10° C. for 1440 minutes under 0.2 Pa; and 5) a secondary drying process at 25° C. for 500 minutes under 0.2 Pa.

Afterward, 3 mL of water for injection was added, resulting in preparing a reconstituted solution containing β-NMN and a hyaluronidase.

TABLE 1

| | Preliminary | | Preliminary | | Preliminary | | Preliminary | | Preliminary | | Preliminary | | Preliminary | | Preliminary | |
|---|---|---|---|---|---|---|---|---|---|---|---|---|---|---|---|---|
| | Example | | Example | | Example | | Example | | Example | | Example | | Example | | Example | |
| | 1 | | 2 | | 3 | | 4 | | 5 | | 6 | | 7 | | 8 | |
| β-NMN | 900 | mg | 900 | mg | 900 | mg | 900 | mg | 900 | mg | 900 | mg | 900 | mg | 900 | mg |
| Hyaluronidase | 300 | IU | 300 | IU | 300 | IU | 300 | IU | 300 | IU | 300 | IU | 300 | IU | 300 | IU |

| | | | | | | | | | | | | | | | |
|---|---|---|---|---|---|---|---|---|---|---|---|---|---|---|---|
| Stabilizer | Mannitol | 300 | mg | 300 | mg | 300 | mg | 300 | mg | 300 | mg | 300 | mg | — | — |

| | | | | | | | | | |
|---|---|---|---|---|---|---|---|---|---|
| Sorbitol | — | — | — | — | — | — | 300 | mg | — |

| | | | | | | | | | |
|---|---|---|---|---|---|---|---|---|---|
| Lactose | — | — | — | — | — | — | — | 300 | mg |

| | | | | | | | | | | |
|---|---|---|---|---|---|---|---|---|---|---|
| Osmotic | HP-gamma- | — | — | 100 | mg | — | — | — | — | — |

| regulator | cyclodextrine | | | | | | | | | |
|---|---|---|---|---|---|---|---|---|---|---|
| | Captisol | — | — | — | 100 | mg | — | — | — | — |

| pH | NaOH | q.s. | q.s. | | q.s. | q.s. | — | — | q.s. | q.s. |
|---|---|---|---|---|---|---|---|---|---|---|
| adjuster | Tromethamine | — | — | | — | — | q.s. | — | — | — |
| (1): | Sodium | — | — | | — | — | — | q.s. | — | — |
| Alkalizing | bicarbonate | | | | | | | | | |
| agent | | | | | | | | | | |
| pH adjuster | HCl | q.s. | q.s. | | q.s. | q.s. | q.s. | q.s. | q.s. | q.s. |
| (2): HCl | | | | | | | | | | |
| Surfactant | Polysorbate | | 10 | mg | | | | | | |

80

To evaluate the short-term stability of the reconstituted solutions containing β-NMN and a hyaluronidase prepared in Preliminary Examples 1 to 8, after 48 hours, the number of insoluble fine particles was measured at 25° C. using a microscopic counting method. The results are shown in Table 2.

TABLE 2

| | | Preliminary | Preliminary | Preliminary | Preliminary | Preliminary | Preliminary | Preliminary | Preliminary |
|---|---|---|---|---|---|---|---|---|---|
| | | Example 1 | Example 2 | Example 3 | Example 4 | Example 5 | Example 6 | Example 7 | Example 8 |
| Insoluble | 10 | 2 | 1 | 0 | 4 | 10 | 25 | 0 | 0 |
| fine | μm | | | | | | | | |
| particles | 25 | 0 | 0 | 0 | 0 | 5 | 6 | 0 | 0 |
| (48 hrs) | μm | | | | | | | | |
| | 50 | 0 | 0 | 0 | 0 | 0 | 0 | 0 | 0 |
| | μm | | | | | | | | |

As shown in Table 2, after 48 hours, the reconstituted solutions containing β-NMN and a hyaluronidase prepared in Preliminary Examples 1 to 8 had generated small amounts of or almost no insoluble fine particles, and therefore are considered to have short-term stability. However, as shown in Preliminary Examples 5 and 6, when tromethamine or sodium bicarbonate was used instead of NaOH as an alkalizing agent, an increase in the number of insoluble fine particles is considered to relatively reduce short-term stability.

### Comparative Examples 1 and 2 and Examples 1 to 4: Preparation of Injectable Formulations (SC Formulation) Using Lyophilized Formulation Containing β-NMN and Hyaluronidase

900 mg of β-NMN and 300 IU of a hyaluronidase (Ningbo Zlinzyme Biosciences, China) were added to 2.7 mL of nitrogen-substituted water for injection, stirred and constituted for 60 minutes. Subsequently, mannitol and captisol were optionally added to constitute for 30 minutes, and then NaOH and/or HCl were/was added q.s. for pH adjustment. The solution was collected up to 3 mL. The collected solution was filtered through a 0.2 μm cellulose acetate (CA) filter. The filtered solution was put into a 20 mL vial, and lyophilized using a lyophilizing apparatus (JABA Lyoph-Pride LP20), thereby preparing a lyophilized formulation for β-NMN injection (water content≤5 (w/w) %). Here, lyophilizing was performed by 1) a loading process at 5° C. under atmospheric pressure; 2) a stabilization process at 5° C. for 120 minutes under atmospheric pressure; 3) a freezing process at −25° C. for 720 minutes under atmospheric pressure; 4) a primary drying process at 10° C. for 1440 minutes under 0.2 Pa; and 5) a secondary drying process at 25° C. for 500 minutes under 0.2 Pa.

Afterward, 3 mL of water for injection was added, resulting in preparing an injectable formulation (SC formulation) containing β-NMN and a hyaluronidase in which the β-NMN concentration is 300 mg/mL, the hyaluronidase concentration is 100 IU/mL, and the pH and osmotic pressure are adjusted, as shown in Table 3.

TABLE 3

| | Comparative | | Comparative | | | | | | | | | |
|---|---|---|---|---|---|---|---|---|---|---|---|---|
| | Example 1 | | Example 2 | | Example 1 | | Example 2 | | Example 3 | | Example 4 | |
| β -NMN | 900 | mg | 900 | mg | 900 | mg | 900 | mg | 900 | mg | 900 | mg |
| Hyaluronidase | 300 | IU | 300 | IU | 300 | IU | 300 | IU | 300 | IU | 300 | IU |

| | | | | | | | | | | | |
|---|---|---|---|---|---|---|---|---|---|---|---|
| Mannitol | 300 | mg | — | 300 | mg | 300 | mg | 300 | mg | 300 | mg |

| | | | | | | |
|---|---|---|---|---|---|---|
| Captisol | — | — | — | — | 100 | mg |

| | | | | | | |
|---|---|---|---|---|---|---|
| NaOH | q.s. | — | q.s. | q.s. | q.s. | — |
| HCl | q.s. | — | q.s. | q.s. | q.s. | q.s. |
| pH | 3.3 | 4 | 6 | 7 | 8 | 6 |
| Osmotic pressure | 1,125 | 720 | 1,130 | 1,115 | 1,133 | 835 |
| (mOsmol/kg) | | | | | | |

To evaluate the long-term stability of the injectable formulations (SC formulations) using the lyophilized formulations containing β-NMN and a hyaluronidase prepared in Comparative Examples 1 and 2 and Examples 1 to 4, the formulations were stored at 25° C. for 8 weeks, and after 8 weeks, the changes in β-NMN content were measured as below. The results are shown in Table 4.
Equipment: LC-PDA (Waters HPLC system)
Conditions: column (GL Sciences InerSustain C18, 250×4.6 mm, 5 μm), mobile phase (10 mM phosphate-buffered saline (pH 3.0)/methanol=90/10), flow rate (1.0 mL/min), injection amount (20 μL)

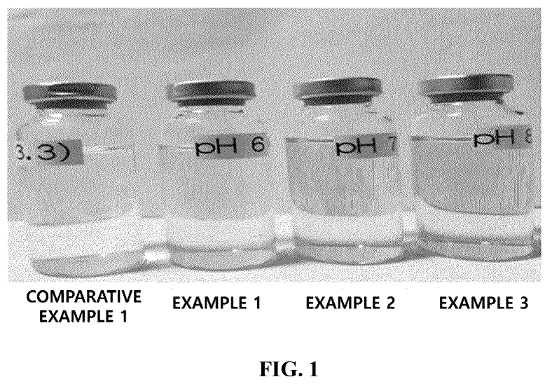

In addition, after 8 weeks, the appearance of the formulations was visually observed. The results are also shown in Table 4 and FIG. 1.

TABLE 4

| | | Comparative | Comparative | | | | |
|---|---|---|---|---|---|---|---|
| Test items | | Example 1 | Example 2 | Example 1 | Example 2 | Example 3 | Example 4 |
| Content | Initial | 99.8 | 98.9 | 97.7 | 98.8 | 101.1 | 97.3 |
| | value | | | | | | |
| | After 8 | 99.7 | 96.7 | 97.9 | 96.2 | 96.7 | 97.7 |
| | weeks | | | | | | |
| Appearance | After 4 | Transparent | Transparent | Transparent | Transparent | Light | Transparent |
| | weeks | | | | | brown | |
| | After 8 | Transparent | Transparent | Transparent | Light brown | Light | Transparent |
| | weeks | | | | | brown | |

As shown in Table 4 and FIG. 1, the injectable formulations (SC formulations) using the lyophilized formulations containing β-NMN and a hyaluronidase prepared in Comparative Examples 1 and 2 and Examples 1 to 4 show a negligible change in β-NMN content at 25° C. for 8 weeks and retain a transparent or light brown color, indicating that the formulations have excellent stability. Meanwhile, compared to Examples 1 and 4, the formulations of Examples 2 and 3 show a greater change in β-NMN content with increasing pH, and are observed to have a light brown color, indicating that they are slightly degraded in stability.

### Comparative Examples 3 to 8: Preparation of Injectable Formulations (SC Formulations) Using Liquid Formulation Containing β-NMN and Hyaluronidase

Injectable formulations (SC formulations) containing β-NMN and a hyaluronidase were prepared in the same manner as in Comparative Examples 1 and 2 and Examples 1 to 4 using a liquid formulation that did not undergo lyophilizing.

To evaluate the long-term stability of the injectable formulations (SC formulations) using the liquid formulation containing β-NMN and a hyaluronidase prepared in Comparative Examples 3 to 8, the formulations were stored at 25° C. for 8 weeks as below, and the changes in β-NMN content after 8 weeks were measured. The results are shown in Table 5.
Equipment: LC-PDA (Waters HPLC system)
Conditions: column (GL Sciences InerSustain C18, 250×4.6 mm, 5 μm), mobile phase (10 mM phosphate-buffered saline (pH 3.0)/methanol=90/10), flow rate (1.0 m/min), injection amount (20 μL)

In addition, after 8 weeks, the appearances of the formulations were visually observed. The results are also shown in Table 5.

TABLE 5

| | | Comparative | Comparative | Comparative | Comparative | Comparative | Comparative |
|---|---|---|---|---|---|---|---|
| Test items | | Example 3 | Example 4 | Example 5 | Example 6 | Example 7 | Example 8 |
| Content | Initial | 98.9 | 100.6 | 99.9 | 99.3 | 99.6 | 101.1 |
| | value | | | | | | |
| | After 8 | 78.2 | 78.2 | 77.9 | 60.1 | 58.3 | 72.9 |
| | weeks | | | | | | |
| Appearance | After 4 | Transparent | Transparent | Transparent | Dark brown | Dark brown | Brown |
| | weeks | | | | | | |
| | After 8 | Transparent | Transparent | Transparent | Dark brown | Dark brown | Brown |
| | weeks | | | | | | |

As shown in Table 5, in the injectable formulations (SC formulations) using the liquid formulation containing β-NMN and a hyaluronidase prepared in Comparative Examples 3 to 8, considerably great changes in β-NMN content were confirmed at 25° C. for 8 weeks, indicating that the stability of the formulations substantially declined.

### Experimental Example 1: Pain Evaluation Experiment in Subcutaneous Administration—Visual Analogue Scale (VAS) Assessment Experiment

When 3 mL each of the injectable formulations using the lyophilized formulation containing β-NMN and a hyaluronidase prepared in Comparative Examples 1 and 2 and Examples 1 to 4 was administered into subcutaneous fat (SC formulation), a pain level was evaluated using VAS. After providing sufficient explanation and obtaining informed consent from 25 voluntary participants, a double-blind trial was conducted. All procedures were performed by a single operator, and both the operator and the voluntary participants were blinded to the components of the injectable formulation. VAS scores were classified on a scale from 0 to 10, with 0 indicating no pain and 10 indicating extremely unbearable pain. The results are shown in Table 6 below.

TABLE 6

| | Comparative | Comparative | Example | Example | Example | Example |
|---|---|---|---|---|---|---|
| | Example 1 | Example 2 | 1 | 2 | 3 | 4 |
| VAS score | 6.2 | 6.8 | 2.8 | 2.4 | 2.6 | 2.1 |

As shown in Table 6, when the formulations in Comparative Examples 1 and 2 were subcutaneously administered, considerable levels of pain were confirmed, which may be attributed to the low pH values. Particularly, in the case of Comparative Example 2 without mannitol as a stabilizer, more severe pain was confirmed. Meanwhile, in the case of Examples 1 to 4, as the pH was maintained to be more than 4 to 8 (particularly, a pH of 6 to 8), the subcutaneous administration is considered to be capable of substantially reducing pain.

### Experimental Example 2: Evaluation of Stability and Pain Upon Injection According to Application of Different Stabilizers in Injectable Formulations Using Lyophilized Formulation Containing β-NMN and Hyaluronidase

### (1) Evaluation of Stability and Pain Upon Injection of Injectable Formulations According to Application of Combination of “Mannitol” as First Stabilizer and Second Stabilizer

TABLE 7

| | Injectable formulation using lyophilized formulation | | | | | | | | | |
|---|---|---|---|---|---|---|---|---|---|---|
| Classification | Example 5 | | Example 6 | | Example 7 | | Example 8 | | Example 9 | |
| β-NMN | 900 | mg | 900 | mg | 900 | mg | 900 | mg | 900 | mg |
| Hyaluronidase | 300 | IU | 300 | IU | 300 | IU | 300 | IU | 300 | IU |

| Stabilizer | | Mannitol 150 | Mannitol 150 | Mannitol 150 | Mannitol 150 | Mannitol 150 |
|---|---|---|---|---|---|---|
| | | mg, | mg, | mg, | mg, | mg, |
| | | Xylitol | Trehalose 150 | Sucrose | Maltose | Glucose |
| | | 150 mg | mg | 150 mg | 150 mg | 150 mg |
| Hydrochloric acid | | q.s. | q.s. | q.s. | q.s. | q.s. |
| Sodium hydroxide | | q.s. | q.s. | q.s. | q.s. | q.s. |
| pH | | pH 6 | pH 6 | pH 6 | pH 6 | pH 6 |
| Osmotic pressure | | 1280 | 1290 | 1280 | 1280 | 1250 |
| (mOsmol/kg) | | | | | | |
| Evaluation | Appearance | Clear solution | Clear solution | Clear solution | Clear solution | Clear solution |
| of stability | right after | | | | | |
| of | redissolution | | | | | |
| injectable | Appearance | Clear solution | Clear solution | Clear solution | Clear solution | Clear solution |
| formulation | at 25° C. | | | | | |
| | after 8 | | | | | |
| | weeks | | | | | |

| | | | | | |
|---|---|---|---|---|---|
| VAS score | 2.4 | 2.3 | 2.1 | 2.2 | 2.1 |

As shown in Table 7, the injectable formulations using a lyophilized formulation according to Examples 5 to 9 are examples in which “mannitol” as a first stabilizer is used in combination with “xylitol, trehalose, sucrose, maltose, or glucose” as a second stabilizer. That is, due to the combination of the first stabilizer (mannitol) and the second stabilizer (xylitol, trehalose, sucrose, maltose, or glucose), the injectable formulations using a lyophilized formulation according to Examples 5 to 9 remain clear solutions at 25° C. for up to 8 weeks, indicating that they can exhibit excellent stability and excellent safety as injections.

Furthermore, the injectable formulations using a lyophilized formulation according to Examples 5 to 9 were able to substantially reduce pain upon injection by optimizing the range of osmotic pressure from 800 mOsmol/kg to 1,600 mOsmol/kg, as confirmed in <Experimental Example 1: Pain evaluation experiment in subcutaneous administration—visual analogue scale (VAS) assessment experiment>.

### (2) Evaluation of Stability and Pain Upon Injection of Injectable Formulations According to Application of Second Stabilizer Alone or in Combination Instead of “Mannitol”

TABLE 8

| | Injectable formulation using lyophilized formulation | | | | | | | | | | | |
|---|---|---|---|---|---|---|---|---|---|---|---|---|
| Classification | Example 10 | | Example 11 | | Example 12 | | Example 13 | | Example 14 | | Example 15 | |
| β-NMN | 900 | mg | 900 | mg | 900 | mg | 900 | mg | 900 | mg | 900 | mg |
| Hyaluronidase | 300 | IU | 300 | IU | 300 | IU | 300 | IU | 300 | IU | 300 | IU |

| Stabilizer | | Xylitol | Trehalose | Sucrose 300 | Maltose | Glucose 300 | Xylitol 150 |
|---|---|---|---|---|---|---|---|
| | | 300 mg | 300 mg | mg | 300 mg | mg | mg, |
| | | | | | | | Sucrose |
| | | | | | | | 150 mg |
| Hydrochloric acid | | q.s. | q.s. | q.s. | q.s. | q.s. | q.s. |
| Sodium hydroxide | | q.s. | q.s. | q.s. | q.s. | q.s. | q.s. |
| pH | | pH 6 | pH 6 | pH 6 | pH 6 | pH 6 | pH 6 |
| Osmotic pressure | | 1290 | 1190 | 1190 | 1180 | 1280 | 1270 |
| (mOsmol/kg) | | | | | | | |
| Evaluation | Appearance | Clear | Clear | Clear | Clear | Clear | Clear |
| of stability | right after | solution | solution | solution | solution | solution | solution |
| of | redissolution | | | | | | |
| injectable | Appearance | Clear | Clear | Clear | Clear | Clear | Clear |
| formulation | at 25° C. | solution | solution | solution | solution | solution | solution |
| | after 8 | | | | | | |
| | weeks | | | | | | |

| | | | | | | |
|---|---|---|---|---|---|---|
| VAS score | 2.4 | 2.3 | 2.5 | 2.6 | 2.5 | 2.4 |

As shown in Table 8, the injectable formulations using a lyophilized formulation according to Examples 10 to 15 are examples in which, instead of mannitol, xylitol, trehalose, sucrose, maltose, or glucose alone is employed, or a combination of xylitol and sucrose is employed. That is, by employing a second stabilizer alone (xylitol, trehalose, sucrose, maltose, or glucose) instead of mannitol, or a combination of xylitol and sucrose as a second stabilizer, the injectable formulations using a lyophilized formulation according to Examples 10 to 15 remain clear solutions at 25° C. for up to 8 weeks, indicating that they can exhibit excellent stability and excellent safety as injections.

Moreover, the injectable formulations using a lyophilized formulation according to Examples 10 to 15 were also able to substantially reduce pain upon injection by optimizing the range of osmotic pressure from 800 mOsmol/kg to 1,600 mOsmol/kg, as confirmed in <Experimental Example 1: Pain evaluation experiment in subcutaneous administration—visual analogue scale (VAS) assessment experiment>.

### Experimental Example 3: Assessment of Blood NAD+/NADH Concentrations in Subcutaneous Administration of Injectable Formulations for Pain Reduction, Including β-NMN and Hyaluronidase

The injectable formulation for pain reduction using a lyophilized formulation of β-NMN and a hyaluronidase prepared in Example 1 was administered into subcutaneous fat of a rat to contain β-NMN at 500 mg/kg/day for 3 days (SC formulation), and after 5, 30, 60, 120, and 360 minutes, the blood was collected. Blood NAD+ and blood NADH concentrations were measured at each time point using an ABCAM NAD+/NADH assay kit (Cat No. ab65348).

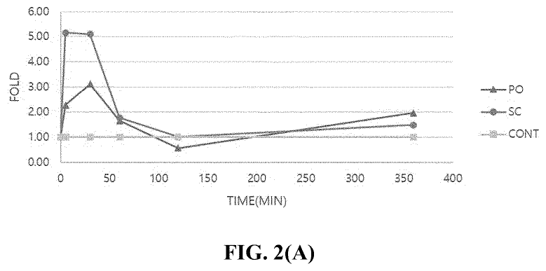

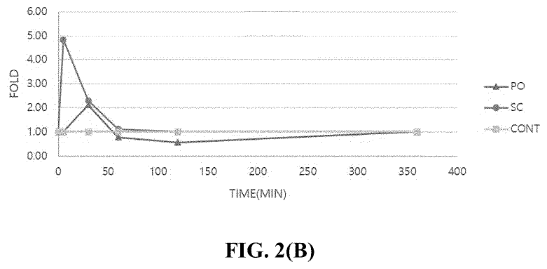

Here, these concentrations were compared with those when the β-NMN-only formulation was orally administered to the rat at a dose of 500 mg/kg/day for 3 days (PO formulation), and the case in which an injectable formulation free of β-NMN and a hyaluronidase as a placebo was intravenously administered to a rat for three days (CONT) was prepared as a control group. The results are shown in Tables 9 and 10 and FIGS. 2(A) and 2(B).

TABLE 9

| | Blood NAD+ concentration | | | | | | | |
|---|---|---|---|---|---|---|---|---|
| | | | | | | | | Initial |
| Classification | 0 min | 5 min | 30 min | 60 min | 120 min | 360 min | Mean | mean |
| SC | 1.00 | 5.16 | 5.10 | 1.76 | 1.00 | 1.47 | 2.90 | 4.01 |
| PO | 1.00 | 2.27 | 3.12 | 1.65 | 0.56 | 1.95 | 1.91 | 2.35 |
| CONT | 1.00 | 1.00 | 1.00 | 1.00 | 1.00 | 1.00 | 1.00 | 1.00 |

As shown in Table 9 and FIG. 2(A), compared to the placebo administration, for the SC formulation, it was confirmed that the mean blood NAD+ concentration shows a substantial increase to 200% to 400% (particularly, 250% to 350%) at each time point, which is understood to be a markedly higher value compared to that of the PO formulation. Especially, at the earlier time points (5 min, 30 min, and 60 min), the mean blood NAD+ concentrations were confirmed to be significantly increased to 350% to 450%, compared to those of the placebo administration. In addition, it is confirmed that the SC formulation has the maximum blood NAD+ concentration at 5 minutes, which is 516% higher than that of the placebo administration.

TABLE 10

| | Blood NADH concentration | | | | | | | |
|---|---|---|---|---|---|---|---|---|
| | | | | | | | | Initial |
| Classification | 0 min | 5 min | 30 min | 60 min | 120 min | 360 min | Mean | mean |
| SC | 1.00 | 4.83 | 2.29 | 1.12 | 1.00 | 1.00 | 2.05 | 2.75 |
| PO | 1.00 | 1.00 | 2.12 | 0.78 | 0.56 | 1.00 | 1.09 | 1.30 |
| CONT | 1.00 | 1.00 | 1.00 | 1.00 | 1.00 | 1.00 | 1.00 | 1.00 |

As shown in Table 10 and FIG. 2(B), compared to the placebo administration, for the SC formulation, the mean blood NADH concentration shows a relative increase to 150% to 300% (particularly, 150% to 250%) at each time point, which is understood to be a markedly higher value than that of the PO formulation. Especially, at the earlier time points (5 min, 30 min, and 60 min), the mean blood NADH concentrations are confirmed to be 250% to 350% higher than those of the placebo administration. In addition, the SC formulation also shows the maximum blood NADH concentration at 5 minutes, which is confirmed to be 483% higher than that of the placebo administration.

Summarizing the above results, compared to the placebo administration, the SC formulation may show that the increments in the mean and maximum blood NAD+ concentrations are relatively higher than the increments in the mean and maximum blood NADH concentrations. That is, as the injectable formulation for pain reduction using a lyophilized formulation containing β-NMN and a hyaluronidase prepared in Example 1 is implemented in the SC formulation, the NAD+/NADH ratio can be optimized in the body, leading to an excellent anti-aging related effect.

An injectable formulation for pain reduction according to the present invention includes β-NMN and a hyaluronidase, and has a pH of more than 4 to 8. Therefore, it has advantages of improving stability and safety and especially minimizing pain induction.

In addition, the injectable formulation for pain reduction according to the present invention can optimize a NAD+/NADH ratio in the body, and thus can have an excellent anti-aging related effect.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive.

1. An injectable formulation for pain reduction upon injection, comprising:
β-nicotinamide mononucleotide (β-NMN) at a concentration of 1 mg/mL to 1,000 mg/mL; and
a hyaluronidase at a concentration of 5 IU/mL to 1,500 IU/mL;
one or more stabilizers selected from the group consisting of mannitol, sorbitol, and lactose; and
one or more alkalizing agents selected from the group consisting of sodium hydroxide (NaOH), calcium hydroxide (Ca(OH)₂), and potassium hydroxide (KOH), or hydrochloric acid (HCl),
wherein the injectable formulation has a pH of greater than 4 and less than 7, and an osmotic pressure of 800 mOsmol/kg to 1,300 mOsmol/kg.

2. (canceled)

3. (canceled)

4. The injectable formulation of claim 1, which is for subcutaneous or intramuscular administration.

5. (canceled)

6. (canceled)

7. (canceled)

8. The injectable formulation of claim 1, wherein the β-NMN is administered daily at a dose of 0.5 mg/kg to 500 mg/kg.

9. The injectable formulation of claim 1, wherein blood is collected at 5, 30, 60, 120, and 360 minutes after administering the injectable formulation and is subjected to analysis using a NAD+/NADH assay kit, revealing that i) the mean blood NAD+ concentration shows a 200% to 400% increase at each time point compared to placebo administration, and ii) the mean blood NADH concentration shows a 150% to 300% increase at each time point compared to placebo administration.

10. A method of preparing an injectable formulation for pain reduction upon injection according to claim 1, comprising:
(a) preparing a solution including β-nicotinamide mononucleotide (β-NMN); a hyaluronidase; one or more stabilizers selected from the group consisting of mannitol, sorbitol, and lactose; and one or more alkalizing agents selected from the group consisting of sodium hydroxide (NaOH), calcium hydroxide (Ca(OH)₂), and potassium hydroxide (KOH), or hydrochloric acid (HCl);
(b) preparing a lyophilized formulation by lyophilizing the solution; and
(c) applying an infusion solution to the lyophilized formulation,
wherein the injectable formulation has a pH greater than 4 and less than 7, and an osmotic pressure of 800 mOsmol/kg to 1.300 mOsmol/kg.

11. (canceled)

12. (canceled)
